(19) United States
(12) Patent Application Publication
(10) Pub. No.: US 2007/0217575 A1
(43) Pub. Date: Sep. 20, 2007

(54) Particle therapy system

(76) Inventors: Werner Kaiser, Langquaid (DE); Vitali Lazarev, Rottenbach (DE)

Correspondence Address: BRINKS HOFER GILSON & LIONE, P.O. BOX 10395, CHICAGO, IL, 60610, US

(21) Appl. No.: 11/652,919

(22) Filed: Jan. 12, 2007

(30) Foreign Application Priority Data
Jan. 20, 2006 (DE) 10 2006 002 908.9

(51) Int. Cl. A61N 5/10; A61B 6/04

(52) U.S. Cl. 378/209; 378/65

(57) ABSTRACT

A particle therapy system is provided. The particle therapy system includes a radiation chamber having a floor with a plurality of movable segments. A rotatable gantry surrounds the radiation chamber. An examination table is positionable inside the radiation chamber. The movable segments are operable to be driven underneath a floor region bordering on the respective movable segment.

The present patent document claims the benefit of the filing date of DE 2006 002 908.9, filed Jan. 20, 2006, which is hereby incorporated by reference.

### BACKGROUND

The present embodiments relate to a particle therapy system.

Particle therapy may be used to treat cancer. In particle therapy, a particle beam, which includes protons or heavy ions, for example, is generated in a suitable accelerator. The particle beam is guided in a radiation conduit and enters a radiation chamber via an exit window of the radiation conduit. Generally, only one local radiation exit window is provided because of the complicated course of the radiation. In some systems, however, a rotatable gantry with an exit window is provided. Because of the complicated radiation course, the gantry is very large in volume. The gantry encloses the approximately cylindrical radiation chamber into which an examination table is moved. For the most precise possible treatment, the patient's tissue to be treated is positioned as near as possible in the isocenter (the target of the beam upon rotation of the gantry) of the system.

At least one radiation detector and passive beam elements are typically disposed in a radiation unit also known as a nozzle. The nozzle is located at the end of the radiation conduit and is directly in front of the exit window. The gantry is ideally rotatable around the patient by 360°, which enables irradiating the patient from below. The radiation unit must also be rotatable in the region below the patient. For that purpose, the floor of the radiation chamber opens and adapts to the rotation of the radiation unit. However, at the same time, a floor in the radiation chamber is needed so that the patient is accessible, maintenance work can be done, and there is no risk of falling for the technicians.

International Patent Disclosure WO 2004/026401 A1 discloses a radiation chamber that is a half-open room-sized space. The floor of this room is solid, except for a slit approximately 50 cm wide for guiding a radiation unit. The slit is covered with a rolling covering that is guided on both ends. The gantry is rotatable by only 180°.

### SUMMARY

The present embodiments may obviate one or more of the limitations or drawbacks inherent in the related art. For example, in one embodiment, a particle therapy system is simple and compact in its construction and is able to irradiate the patient from below.

In one embodiment, a particle therapy system includes a rotatable gantry, which surrounds a radiation chamber having a floor that has a plurality of movable segments. An examination table may be positioned in the radiation chamber. The movable segments are drivable underneath an adjoining floor region.

In one embodiment, the movable segments have a platelike form, so that only a simple load-bearing construction and little technological effort and expense for moving the segments are required. In one embodiment, the movable segments are driven underneath the adjoining floor region. In this embodiment, the segments moved away are supported under the adjoining floor region in such a way that they neither interfere with the rotation of the gantry nor occupy space above the floor. In this embodiment, driving of the segments is technologically easy to achieve, and very little space is needed to accommodate the segments that are removed from the floor.

In one embodiment, only those segments where there is a risk of collision with the radiation unit are removed. The individually movable segments make the floor in the radiation chamber accessible to people over a large area. The floor in the radiation chamber is embodied such that driving one or more segments has no adverse effects on the strength of the remaining floor. In one embodiment, the movable segments that extend in the longitudinal direction of the cylindrical radiation chamber may be driven in the longitudinal direction. Alternatively, the movable segments may be driven laterally, for example, underneath an adjacent segment.

In one embodiment, driving the segments includes lowering the segments and thrusting them into a void under the adjoining floor region. Since a plurality of segments are provided, only individual portions of the floor ever have to be opened in order to enable satisfactory rotation of the protruding radiation unit or radiation of the patient from below the examination table. This embodiment minimizes the opening in the radiation chamber floor, which is partly covered by the radiation unit. In this embodiment, the floor adjacent to the examination table can be walked on at all times, without relatively large regions of the floor being open. Safe and secure access to the patient is thus assured.

In one embodiment, the floor of the radiation chamber is independent of the radiation chamber and of the gantry and is not connected to them. The load-bearing construction and the movement mechanism of the segments are mounted outside the radiation chamber. In this embodiment, the construction of the radiation chamber is simple, for example, the side wall and back wall rotate with the radiation chamber but the movable floor remains stationary.

In one embodiment, the floor segments may be driven underneath a solid floor adjacent to the radiation chamber. The solid floor is a foundation, for example, a firm foundation, which is formed on the order of a plate of a predetermined thickness. In this embodiment, the individual movable segments are driven underneath the solid floor, which opens the movable floor. The movable segments may be driven all the way out of the radiation chamber. In this embodiment, a large amount of space is reserved for the satisfactory rotation of the gantry.

In one embodiment, the movable segments are pivotable about a pivot axis and can be driven in the pivoted position. For example, when driving is not possible in the horizontal position of the segments because of space reasons, the movable segments may be pivoted.

In one embodiment, the segments are pivotable substantially by 90°. In this embodiment, the strength of the floor is maintained, even after the movable segments are rotated 90° into the vertical position because the floor is hollow only in small partial regions.

In one embodiment, the solid floor includes recesses for the segments. The recesses in the solid floor are especially suitable for a thick solid floor. The segments are therefore driven into the solid floor without long driving distances. The floor segments may be driven all way into the recesses. The recesses may have a slitlike form, and the segments are thrust inward in a horizontal position, or the recesses have a gap for receiving the pivoted segments. It is also possible for a plurality of segments to be accommodated in one recess, or for only one recess that receives all the segments to be provided. The shape, size and position of the recesses depend in principle on the thickness of the solid floor and on the demands made in terms of its strength and stability.

In one embodiment, the recesses include vertical chutes. These chutes have a width that is essentially equivalent to the thickness of the movable segments. The movable segments are driven into the recesses in the vertical position after being pivoted by 90°. This embodiment of the recesses offers stability of the solid floor.

In one embodiment, the pivot axes are disposed such that upon rotation of the segments, adequate lowering for driving into the recesses is assured. This lowered position makes it possible to drive the segments underneath or into the solid floor. The recesses here are disposed under the surface of the solid floor, so that a sufficiently thick floor covering above the recesses assures high stability of the solid floor.

In one embodiment, the movable segments are each rotatable about the pivot axis by a respective lever arm. The lever arm has a simple structure. In one embodiment, the lever arm is able to lower and pivot the movable segments.

In one embodiment, each of the lever arms is expediently disposed on a peripheral side of the movable segments, which allows combined motion of the segments.

In one embodiment, the gantry is rotatable by 360°. In this embodiment, the particle beam can be aimed at arbitrary angles, for example, at the diseased tissue of the patient.

In one embodiment, the radiation therapy system includes a support arm secured to the solid floor. The support arm is able to place robotically the examination table inside the radiation chamber. In this embodiment, the examination table can be driven into the radiation chamber surrounded by the gantry and held there, without the examination table having contact with the movable floor. In this embodiment, the examination table has an increased stability because its position in the radiation chamber does not depend on how the various segments are disposed.

In one embodiment, the radiation therapy system includes a mechanical drive that drives the floor segments. In this embodiment, the radiation system has a high degree of automation, which leads to precision of the treatment.

In one embodiment, the radiation therapy system includes a control unit. The control unit automatically drives the individual movable segments as a function of the position of the gantry. When the radiation unit reaches the movable floor, for example, one or more segments in the peripheral region of the floor is automatically moved away, so that the radiation unit can continue to be rotated. In one embodiment, the control unit is able to remove the movable segments in the middle region of the floor as needed. In this embodiment, the particle beam is able to reach the patient, even from below the examination table.

### BRIEF DESCRIPTION OF THE DRAWINGS

FIG. 1 is a perspective view that shows one embodiment of a particle therapy system;

FIG. 2 is a view from a radiation chamber of the particle therapy system of one embodiment of an open side of the radiation chamber; and

FIG. 3 is a top view that shows one embodiment of a section through the radiation chamber.

### DETAILED DESCRIPTION

In the drawings, the same references all have same meanings.

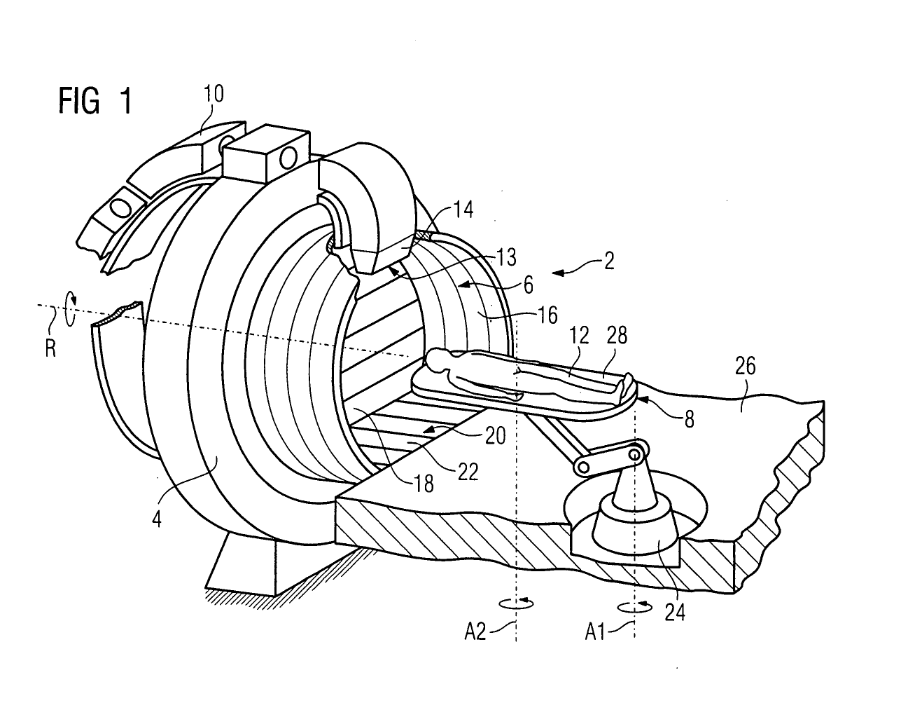

In one embodiment, as shown in FIG. 1, a particle therapy system 2 includes a gantry 4 that is rotatable about an axis R of rotation. The gantry 4 surrounds an approximately cylindrical radiation chamber 6, in which an examination table 8 can be positioned. As shown in FIG. 1 in an exploded view, the gantry 4 includes a radiation conduit 10. The radiation conduit 10 guides a particle beam (not shown), such as a heavy ion or proton beam, to treat a patient 12 lying on the examination table 8. The particle beam enters the radiation chamber 6 via an exit window 13 of a radiation unit 14. In one embodiment, the radiation unit 14 is installed in the radiation conduit 10 and protrudes from a rotatable side wall 16 of the radiation chamber 6.

In one embodiment, the side wall 16 includes a simple lining without mechanical load-bearing capability. The rear of the radiation chamber 16 is bounded by a back wall 18. The back wall 18 is a simple lining without mechanical load-bearing capability and rotates with the gantry 4 about the axis R of rotation.

In one embodiment, the radiation chamber 6 includes a floor 20, which has a plurality of movable segments 22. The floor 20 will hereinafter also be called the movable floor.

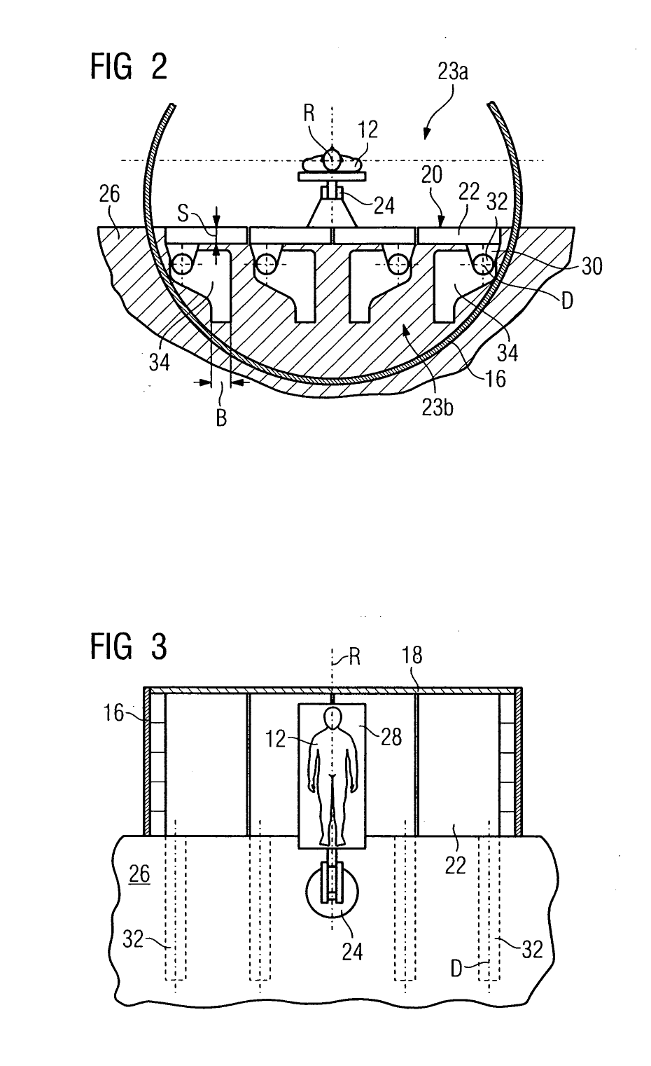

In this exemplary embodiment, “under the radiation chamber 6” is the cylindrical space that is surrounded by the gantry 4 and is bounded laterally by a cylindrical side wall 16 and to the rear by a back wall 18 and is partly open toward the front. As shown in FIG. 2, the movable floor 20 divides the radiation chamber 6 into one upper part 23a and one lower part 23b. The upper part 23a is open toward the front, so that the examination table 8 can be driven into it. The lower part 23 includes a movement mechanism for the segments 22.

In one embodiment, the examination table 8 is driven into the radiation chamber 6 by a patient handling system, for example, a triggered robotic support arm 24. The examination table 8 has no contact with the movable floor 20. In one embodiment, the support arm 24 is mounted on a solid floor 26 of a predetermined thickness that borders the radiation chamber 6 and is located at the same level as the movable floor 20. Alternatively, the solid floor 26 may also be embodied somewhat higher than the movable floor 20.

In one embodiment, the support arm 24 is a multi-axial industrial robot with a multiple-piece mechanism. The support arm 24 may be used to move the examination table 8 translationally in both the horizontal and the vertical directions. In one embodiment, the support arm 24 is rotatable about an axis A1, which is perpendicular to the solid floor 26. The examination table 8 may be rotatable about an axis A2 that extends perpendicular to a tabletop 28 of the examination table 8.

In one embodiment, the support arm 24 is embodied such that the motion of the examination table 8 includes 3° of translational freedom as well as 3° of rotational freedom. The position and distance of the patient 12 from the radiation unit 14 are adjusted using the translational and rotary motions of the examination table 8. In one embodiment, the tabletop 28 remains in a horizontal position during the positioning of the examination table 8 in the radiation chamber 6. In this embodiment, the patient 12 lies stably on the examination table 8 during the positioning of the examination table 8.

FIG. 2 shows the construction of the moveable floor 20 and the solid floor 26. The floor segments 22 have a thickness S, which allows the movable floor 20 to be sufficiently stable when loaded with the weight of at least one person. In one embodiment, each of the segments 22 of the movable floor 20 is rotatable essentially by 90° about an axis D via a peripherally mounted lever arm 30 and a rotatable guide 32. In this embodiment, the segments 22 are lowered upon a rotary motion, so that their top side is located below the surface of the solid floor 26. No additional translational vertical reciprocating motion then takes place.

In this lowered position, the segments 22 are driven into various hollow recesses 34, which are provided in the solid floor 26. The recesses 34 are approximately the same shape and size as the segments 22 with the associated movement mechanism (i.e. the lever arm 30 and the rotatable guides 32). The recesses 34 include vertical chutes with a widened area for the movement mechanism 30, 32. The width B of the vertical chutes 34 is approximately equivalent to the thickness S of the segments 22, plus a tolerance range.

In one embodiment, the recesses 34 are disposed at a suitable distance from the surface of the solid floor 26. The recesses 34 are covered by a floor covering, which assures a high load-bearing capacity of the solid floor 26, even in the regions above the recesses. The thickness of this floor covering or the load-bearing capacity of the solid floor 26 may be increased by making the solid floor 26 higher than the movable floor 20 in the radiation chamber 6. The height above the movable floor 20 may be any suitable distance, for example, on the order of a stairstep.

In one embodiment, the patient 12 is immobilized on the examination table 8 and positioned inside the radiation chamber 6 before performing the therapy. For example, the examination table 8 is driven into the radiation chamber 6 of the gantry via the support arm 24 and positioned such that the diseased tissue of the patient 12 is located in the isocenter of the gantry 4. The gantry 4 is rotated about its axis R of rotation to adjust to an angle that is most favorable for the irradiation. The radiation unit 14 that protrudes from the side wall 16 rotates, and the side wall 16 and the back wall 18 of the radiation chamber 6 rotate together. No relative motion of the radiation unit 14 with respect to the side wall 16 takes place.

There is a risk of collision between the radiation unit 14 and the floor 20 in the peripheral region of the movable floor 20 and below the movable floor 20 because of the relatively large volume of the radiation unit 14. In one embodiment, collision is avoided by a control unit not shown in further detail here. In this embodiment, the control unit drives the individual segments 22 of the movable floor 20 into the recesses 34 in the solid floor 26 as a function of the position of the radiation unit 14, which creates an opening in part of the movable floor 20. The opening in the movable floor 20 is at least partly covered here by the radiation unit 14. This covering reduces the risk of the technicians or other objects located on the movable floor 20 from falling through the openings. In one embodiment, a virtually gap-free floor 22 with a minimal risk of falling is achieved using a skillful choice of the contour and the paths of motion of the segments 22.

In one embodiment, the movable floor 20 is triggered in particular in such a way that with a stationary gantry 4, the access to the patient is assured via at least one segment 22. In one embodiment, the segments 22 in the middle region of the floor 20 may be removed, for radiating the patient 12 from below.

In one embodiment, the construction and driving of one or more movable segments 22 underneath an adjoining floor region, for example, underneath the solid floor 26, allows the patient 12 to be irradiated from below. In this embodiment, the accessibility to the patient 12 is assured at all times. Because the movable segments are driven underneath the floor, they are not a hindrance, and the floor continues to be free for access to the technicians and for perfect positioning of the examination table 8.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A particle therapy system comprising:
a radiation chamber having a floor with a plurality of movable segments;
a rotatable gantry that surrounds the radiation chamber; and
an examination table which is positionable inside the radiation chamber,
wherein at least one of the movable segments is operable to be driven underneath a floor region bordering on the respective movable segment.

2. The particle therapy system as defined by claim 1, wherein the at least one of the movable segments is operable to be driven underneath a solid floor that borders the radiation chamber.

3. The particle therapy system as defined by claim 1, wherein the at least one of the movable segments is pivotable about a pivot axis and can be driven in a pivoted position.

4. The particle therapy system as defined by claim 3, wherein the at least one of the movable segments is pivotable by about 90°.

5. The particle therapy system as defined by claim 2, comprising at least one recess for the at least one of the movable segments embodied in the solid floor.

6. The particle therapy system as defined by claim 5, wherein the at least one recess comprises a vertical chute.

7. The particle therapy system as defined by claim 3, wherein the pivot axis and the at least one of the movable segments are operable to rotate to provide adequate lowering.

8. The particle therapy system as defined by claim 7, wherein the at least one of the movable segments is rotatable about the pivot axis using a lever arm.

9. The particle therapy system as defined by claim 8, wherein the lever arm is disposed on a peripheral side of the at least one of the movable segments.

10. The particle therapy system as defined by claim 1, wherein the gantry is rotatable by 360°.

11. The particle therapy system as defined by claim 2, wherein a support arm secured to the solid floor is operable to place the examination table inside the radiation chamber.

12. The particle therapy system as defined claim 2, comprising a mechanical drive operable to drive the at least one of the movable segments.

13. The particle therapy system as defined by claim 12, comprising a control unit, which automatically drives the at least one of the movable segments as a function of the position of the gantry.

14. The particle therapy system as defined by claim 13 wherein the control unit is operable to automatically drive each of the moveable segments.

15. The particle therapy system as defined by claim 2, wherein the solid floor includes one recess for the plurality of movable segments.

16. The particle therapy system as defined by claim 6, wherein the vertical chute has a width that is approximately equal to the width of the movable segments.
